(19) United States
(12) Patent Application Publication
(10) Pub. No.: US 2022/0387468 A1
(43) Pub. Date: Dec. 8, 2022

(54) METHODS OF TREATING CANCER

(71) Applicant: ASTRAZENECA AB, Sodertalje (SE)

(72) Inventors: Elisabetta LEO, Cambridge (GB); Claudia WINKLER, Cambridge (GB); Mark James O'CONNOR, Cambridge (GB); Gemma Nicole JONES, Cambridge (GB); Andrew James PIERCE, Cambridge (GB)

(21) Appl. No.: 17/771,159

(22) PCT Filed: Oct. 23, 2020
(86) PCT No.: PCT/EP2020/079856

§ 371 Date: Apr. 22, 2022

Related U.S. Application Data
(60) Provisional application No. 62/926,055, filed on Oct. 25, 2019.

(51) Int. Cl. A61K 31/7068; A61K 47/68; A61K 31/519; A61P 35/00; A61K 33/243; A61K 31/17; A61K 31/35; A61P 15/00; G01N 33/574

(52) U.S. Cl. CPC A61K 31/7068; A61K 47/6855; A61K 31/519; A61P 35/00; A61K 33/243; A61K 31/17; A61K 31/35; A61P 15/00; G01N 33/57484; G01N 2800/52

(57) ABSTRACT

Methods of treating cancer with a combination of a WEE1 inhibitor and a DNA-damaging agent in patients having SLFN11-deficient cancer cells are disclosed herein.

### FIELD

The instant disclosure generally relates to methods of treating cancer.

### BACKGROUND

WEE1 is a nuclear kinase that belongs to the serine/threonine family of protein kinases. WEE1 inhibits cyclin-dependent kinases (CDKs) by phosphorylating CDKs on two different sites (Tyr15 and Thr14). WEE1 therefore plays a role in regulating mitotic entry and initiation of DNA replication, cell size, and DNA damage checkpoints. Inhibitors of WEE1 have been tested for the treatment of cancer as monotherapy and in combination with other cancer treatments.

Schlafen 11 (SLFN11) belongs to the Schlafen family of proteins and is only expressed in humans and some primates. Inactivation of SLFN11 in cancer cells has been shown to result in resistance to anticancer agents that cause DNA damage and replication stress. Thus, SLFN11 is a determinant of sensitivity to different classes of DNA-damaging agents and PARP inhibitors. See Zoppoli et al., PNAS 2012; 109: 15030-35; Murai et al., Oncotarget 2016; 7: 76534-50; Murai et al., Mol. Cell 2018; 69: 371-84.

A number of cancer treatments have been developed and approved. However, some cancer treatments are only effective in a fraction of patients. Moreover, a fraction of cancer patients become resistant to certain cancer treatments. Thus, a need exists for methods of identifying patients that are responsive to cancer treatments so that the cancer treatments can be targeted to appropriate patients. In addition, a need exists for methods of reversing resistance to cancer treatments that is observed in some patients.

### BRIEF SUMMARY

The foregoing needs are met by the methods described herein. In particular, disclosed herein is a method of treating cancer in a patient comprising: a) selecting a patient diagnosed with cancer; b) determining whether the patient's cancer cells are SLFN11-deficient; and, c) if the patient's cancer cells are SLFN11-deficient, co-administering a WEE1 inhibitor and a DNA-damaging agent to the patient. In some embodiments, the patient's cancer cells are negative for SLFN11 expression.

In some embodiments, disclosed herein is a method of treating cancer in a patient comprising: a) selecting a patient diagnosed with cancer; b) determining whether SLFN11 expression is lower in the patient's cancer cells relative to the patient's SLFN11-expressing non-cancer cells; and, c) if SLFN11 expression is lower in the patient's cancer cells relative to the patient's SLFN11-expressing non-cancer cells, co-administering a WEE1 inhibitor and a DNA-damaging agent to the patient. In some embodiments, the patient's cancer cells are negative for SLFN11 expression.

In some embodiments, disclosed herein is a method of treating cancer in a patient comprising: a) selecting a patient diagnosed with cancer; b) determining the expression level of SLFN11 in the patient's cancer cells; and, c) if the expression level of SLFN11 is <10%, co-administering a WEE1 inhibitor and a DNA-damaging agent to the patient. In some embodiments, the expression level of SLFN11 is 0%.

In some embodiments, disclosed herein is a method of treating cancer in a patient that is resistant to treatment with a DNA-damaging agent, comprising: a) determining whether the patient's cancer cells are SLFN11-deficient; and, b) if the patient's cancer cells are SLFN11-deficient, co-administering a WEE1 inhibitor with the DNA-damaging agent to the patient. In some embodiments, the patient's cancer cells are negative for SLFN11 expression.

In some embodiments, disclosed herein is a method of treating cancer in a patient that is resistant to treatment with a DNA-damaging agent, comprising: a) determining whether SLFN11 expression is lower in the patient's cancer cells relative to the patient's SLFN11-expressing non-cancer cells; and, b) if SLFN11 expression is lower in the patient's cancer cells relative to the patient's SLFN11-expressing non-cancer cells, co-administering a WEE1 inhibitor with the DNA-damaging agent to the patient. In some embodiments, the patient's cancer cells are negative for SLFN11 expression.

In some embodiments, disclosed herein is a method of treating cancer in a patient that is resistant to treatment with a DNA-damaging agent, comprising: a) determining the expression level of SLFN11 in the patient's cancer cells; and, b) if the expression level of SLFN11 is <10%, co-administering a WEE1 inhibitor with the DNA-damaging agent to the patient. In some embodiments, the expression level of SLFN11 is 0%.

In some embodiments, the expression level of SLFN11 is determined by immunohistochemistry, mass spectrometry, in-situ hybridization, NanoString, reverse transcription quantitative polymerase chain reaction (RT-qPCR), microarray analysis, bisulfite sequencing, or quantitative methylation-specific polymerase chain reaction (Q-MSP). In a specific embodiment, the expression level of SLFN11 is determined by immunohistochemistry.

In some embodiments of the methods disclosed herein, the cancer is selected from the group consisting of pancreatic cancer, endometrial cancer, ovarian cancer, melanoma, lung cancer, colorectal cancer, colon cancer, rectal cancer, prostate cancer, breast cancer, brain cancer, cervicocerebral cancer, esophageal cancer, thyroid cancer, stomach cancer, gallbladder cancer, liver cancer, choriocarcinoma, uterus body cancer, uterocervical cancer, kidney cancer, bladder cancer, testicular cancer, skin cancer, neuroblastoma, osteosarcoma, Ewing's sarcoma, leukemia, Hodgkin's lymphoma, acute myeloid leukemia, diffuse large B-cell lymphoma, and head and neck cancer.

In some embodiments of the methods disclosed herein, the DNA-damaging agent is selected from the group consisting of gemcitabine, etoposide, cisplatin, carboplatin, oxaliplatin, picoplatin, methotrexate, doxorubicin, daunorubicin, 5-fluorouracil, irinotecan, mitomycin, temozolomide, topotecan, camptothecin, epirubicin, idarubicin, trabectedin, capecitabine, bendamustine, fludarabine, hydroxyurea, trastuzumab deruxtecan, and pharmaceutically acceptable salts thereof.

In some embodiments of the methods disclosed herein, the WEE1 inhibitor is adavosertib or a pharmaceutically acceptable salt thereof.

### BRIEF DESCRIPTION OF THE DRAWINGS

FIG. 1A shows positive and negative staining from the SLFN11 immunohistochemistry (IHC) assay in DU145 xenograft (SLFN11-proficient) and HT29 xenograft tissue (SLFN11-deficient), respectively.

FIG. 2A shows immunoblots for SLFN11 and GAPDH in SLFN11 wild-type (WT) and knockout (KO) DU145 isogenic cells. KO 1 and KO 2 were two different CRISPR-KO clones.

FIG. 2B shows synergy scores (Loewe) resulting from treatment of wild-type SLFN11 (WT) or SLFN11 knockout DU145 cell lines (KO1 and K02) with a combination of gemcitabine (Gem.) and adavosertib.

FIG. 2C shows synergy scores (Loewe) resulting from treatment of wild-type SLFN11 (WT) or SLFN11 knockout DU145 cell lines (KO1 and K02) with etoposide (ETP) and adavosertib.

FIG. 2D shows survival curves of the indicated DNA damaging agents (gemcitabine, etoposide, camptothecin, cisplatin, and hydroxyurea) in the absence or presence of 0.36 μM adavosertib in DU145 isogenic cells.

FIG. 3A shows log IC₅₀ values of gemcitabine monotherapy in a panel of pancreatic cell lines that are either SLFN11-deficient or SLFN11-proficient.

FIG. 3B shows log IC₅₀ values of adavosertib monotherapy in a panel of pancreatic cell lines that are either SLFN11-deficient or SLFN11-proficient.

FIG. 3C shows synergy scores for the combination of gemcitabine and adavosertib in a panel of pancreatic cell lines that are either SLFN11-deficient or SLFN11-proficient.

### DETAILED DESCRIPTION

While embodiments of the invention are shown and described herein, it will be apparent to those skilled in the art that such embodiments are provided by way of example only. Numerous variations, changes, and substitutions will occur to those skilled in the art without departing from the invention. It should be understood that various alternatives to the embodiments described herein may be employed. The section headings used herein are for organizational purposes only and are not to be construed as limiting the subject matter described.

### Definitions

The terms “treat,” “treating,” or “treatment,” and other grammatical equivalents as used herein, include alleviating, abating or ameliorating a disease or condition or one or more symptoms thereof, ameliorating the underlying metabolic causes of symptoms, inhibiting the disease or condition, relieving the disease or condition, causing regression of the disease or condition, relieving a condition caused by the disease or condition, or stopping the symptoms of the disease or condition.

The terms “administer,” “administering,” “administration,” and their grammatical equivalents, as used herein, refer to the methods used to deliver pharmaceutical compositions disclosed herein to the desired site of biological action.

The terms “co-administer,” “co-administration,” “administered in combination with” and their grammatical equivalents, as used herein, are meant to encompass administration of the active agents to a single individual, and, unless specified otherwise, include treatment regimens in which the agents are administered by the same or different route of administration or at the same or different times. They include simultaneous administration in separate compositions, administration at different times in separate compositions, or administration in a composition in which one or more active agents are present.

The term “pharmaceutically acceptable,” as used herein, refers to a material, such as a carrier or diluent, which does not abrogate the biological activity or properties of the active agent, and is relatively nontoxic, i.e., the material may be administered to an individual without causing undesirable biological effects or interacting in a deleterious manner with any of the components of the composition in which it is contained.

The term “pharmaceutically acceptable salt,” as used herein, refers to salts that retain the biological efficacy of the free acid or base of the active agent and that are not biologically or otherwise undesirable. The active agents may react with inorganic or organic bases, or inorganic or organic acids, to form a pharmaceutically acceptable salt. These salts can be prepared in situ during the final isolation and purification, or separately by reacting the purified compounds with a suitable inorganic or organic base, or inorganic or organic acid, and isolating the salt thus formed.

The terms “patient,” “subject,” and “individual” are used interchangeably herein. As used herein, they refer to humans suffering from cancer.

As used herein, the term “the expression level of SLFN11 is” some amount, e.g. 0%, means that the stated amount of cancer cells in the patient's cancer tissue express SLFN11. Similarly, as used herein, the term “the expression level of SLFN11 is <” some amount, e.g. 10%, means that less than the stated amount of cancer cells in the patient's cancer tissue express SLFN11.

As used herein, the term “SLFN11-deficient” refers to an expression level of SLFN11 in the relevant patient, animal, tissue, cell, etc. that is inadequate to exhibit the normal phenotype associated with the gene, or for the protein to exhibit its physiological function. In the context of preclinical models, cells or animals in which the SLFN11 gene is knocked out (KO) are examples of “SLFN11-deficient.”

As used herein, the term “SLFN-11 proficient” refers to an expression level of SLFN11 in the relevant patient, animal, tissue, cell, etc. that is adequate to exhibit the normal phenotype associated with the gene, or for the protein to exhibit its physiological function. In the context of preclinical models, cells or animals in which the SLFN11 gene is expressed at normal levels, i.e., wild-type (WT) cells or animals, are examples of “SLFN11-proficient.”

### Methods of Treatment

In some embodiments, disclosed herein is a method of treating cancer in a patient comprising: a) selecting a patient diagnosed with cancer; b) determining whether the patient's cancer cells are SLFN11-deficient; and, c) if the patient's cancer cells are SLFN11-deficient, co-administering a WEE1 inhibitor and a DNA-damaging agent to the patient. In some embodiments, the patient's cancer cells are negative for SLFN11 expression.

In some embodiments, disclosed herein is a method of treating cancer in a patient comprising: a) selecting a patient diagnosed with cancer; b) determining whether SLFN11 expression is lower in the patient's cancer cells relative to the patient's SLFN11-expressing non-cancer cells; and, c) if SLFN11 expression is lower in the patient's cancer cells relative to the patient's SLFN11-expressing non-cancer cells, co-administering a WEE1 inhibitor and a DNA-damaging agent to the patient. In some embodiments, the patient's cancer cells are negative for SLFN11 expression.

In some embodiments, disclosed herein is a method of treating cancer in a patient comprising: a) selecting a patient diagnosed with cancer; b) determining the expression level of SLFN11 in the patient's cancer cells; and, c) if the expression level of SLFN11 is <25%, co-administering a WEE1 inhibitor and a DNA-damaging agent to the patient. In some embodiments, disclosed herein is a method of treating cancer in a patient comprising: a) selecting a patient diagnosed with cancer; b) determining the expression level of SLFN11 in the patient's cancer cells; and, c) if the expression level of SLFN11 is <20%, co-administering a WEE1 inhibitor and a DNA-damaging agent to the patient. In some embodiments, disclosed herein is a method of treating cancer in a patient comprising: a) selecting a patient diagnosed with cancer; b) determining the expression level of SLFN11 in the patient's cancer cells; and, c) if the expression level of SLFN11 is <15%, co-administering a WEE1 inhibitor and a DNA-damaging agent to the patient. In some embodiments, disclosed herein is a method of treating cancer in a patient comprising: a) selecting a patient diagnosed with cancer; b) determining the expression level of SLFN11 in the patient's cancer cells; and, c) if the expression level of SLFN11 is <10%, co-administering a WEE1 inhibitor and a DNA-damaging agent to the patient. In some embodiments, a WEE1 inhibitor and a DNA-damaging agent are co-administered if the expression level of SLFN11 is <9%. In some embodiments, a WEE1 inhibitor and a DNA-damaging agent are co-administered if the expression level of SLFN11 is <8%. In some embodiments, a WEE1 inhibitor and a DNA-damaging agent are co-administered if the expression level of SLFN11 is <7%. In some embodiments, a WEE1 inhibitor and a DNA-damaging agent are co-administered if the expression level of SLFN11 is <6%. In some embodiments, a WEE1 inhibitor and a DNA-damaging agent are co-administered if the expression level of SLFN11 is <5%. In some embodiments, a WEE1 inhibitor and a DNA-damaging agent are co-administered if the expression level of SLFN11 is <4%. In some embodiments, a WEE1 inhibitor and a DNA-damaging agent are co-administered if the expression level of SLFN11 is <3%. In some embodiments, a WEE1 inhibitor and a DNA-damaging agent are co-administered if the expression level of SLFN11 is <2%. In some embodiments, a WEE1 inhibitor and a DNA-damaging agent are co-administered if the expression level of SLFN11 is <1%. In some embodiments, a WEE1 inhibitor and a DNA-damaging agent are co-administered if the expression level of SLFN11 is 0%.

In some embodiments, disclosed herein is a method of treating cancer in a patient that is resistant to treatment with a DNA-damaging agent, comprising: a) determining whether the patient's cancer cells are SLFN11-deficient; and, b) if the patient's cancer cells are SLFN11-deficient, co-administering a WEE1 inhibitor with the DNA-damaging agent to the patient. In some embodiments, the patient's cancer cells are negative for SLFN11 expression.

In some embodiments, disclosed herein is a method of treating cancer in a patient that is resistant to treatment with a DNA-damaging agent, comprising: a) determining whether SLFN11 expression is lower in the patient's cancer cells relative to the patient's SLFN11-expressing non-cancer cells; and, b) if SLFN11 expression is lower in the patient's cancer cells relative to the patient's SLFN11-expressing non-cancer cells, co-administering a WEE1 inhibitor with the DNA-damaging agent to the patient. In some embodiments, the patient's cancer cells are negative for SLFN11 expression.

In some embodiments, disclosed herein is a method of treating cancer in a patient that is resistant to treatment with a DNA-damaging agent, comprising: a) determining the expression level of SLFN11 in the patient's cancer cells; and, b) if the expression level of SLFN11 is <25%, co-administering a WEE1 inhibitor and a DNA-damaging agent to the patient. In some embodiments, disclosed herein is a method of treating cancer in a patient that is resistant to treatment with a DNA-damaging agent, comprising: a) determining the expression level of SLFN11 in the patient's cancer cells; and, b) if the expression level of SLFN11 is <20%, co-administering a WEE1 inhibitor and a DNA-damaging agent to the patient. In some embodiments, disclosed herein is a method of treating cancer in a patient that is resistant to treatment with a DNA-damaging agent, comprising: a) determining the expression level of SLFN11 in the patient's cancer cells; and, b) if the expression level of SLFN11 is <15%, co-administering a WEE1 inhibitor and a DNA-damaging agent to the patient. In some embodiments, disclosed herein is a method of treating cancer in a patient that is resistant to treatment with a DNA-damaging agent, comprising: a) determining the expression level of SLFN11 in the patient's cancer cells; and, b) if the expression level of SLFN11 is <10%, co-administering a WEE1 inhibitor and a DNA-damaging agent to the patient. In some embodiments, a WEE1 inhibitor and a DNA-damaging agent are co-administered if the expression level of SLFN11 is <9%. In some embodiments, a WEE1 inhibitor and a DNA-damaging agent are co-administered if the expression level of SLFN11 is <8%. In some embodiments, a WEE1 inhibitor and a DNA-damaging agent are co-administered if the expression level of SLFN11 is <7%. In some embodiments, a WEE1 inhibitor and a DNA-damaging agent are co-administered if the expression level of SLFN11 is <6%. In some embodiments, a WEE1 inhibitor and a DNA-damaging agent are co-administered if the expression level of SLFN11 is <5%. In some embodiments, a WEE1 inhibitor and a DNA-damaging agent are co-administered if the expression level of SLFN11 is <4%. In some embodiments, a WEE1 inhibitor and a DNA-damaging agent are co-administered if the expression level of SLFN11 is <3%. In some embodiments, a WEE1 inhibitor and a DNA-damaging agent are co-administered if the expression level of SLFN11 is <2%. In some embodiments, a WEE1 inhibitor and a DNA-damaging agent are co-administered if the expression level of SLFN11 is <1%. In some embodiments, a WEE1 inhibitor and a DNA-damaging agent are co-administered if the expression level of SLFN11 is 0%.

In the methods disclosed herein, the expression level of SLFN11 may be determined by any suitable method known to those of ordinary skill in the art. In some embodiments, the expression level of SLFN11 is determined by mRNA transcript levels or DNA promoter hypermethylation. In some embodiments, the expression level of SLFN11 is determined by immunohistochemistry, mass spectrometry, in-situ hybridization, NanoString, reverse transcription quantitative polymerase chain reaction (RT-qPCR), microarray analysis, bisulfite sequencing, or quantitative methylation-specific polymerase chain reaction (Q-MSP). In a specific embodiment, the expression level of SLFN11 is determined by immunohistochemistry (IHC).

### Diseases

The methods described herein are applicable to the treatment of a variety of cancers. In some embodiments, the cancer is selected from the group consisting of pancreatic cancer, endometrial cancer, ovarian cancer, melanoma, lung cancer, colorectal cancer, colon cancer, rectal cancer, prostate cancer, breast cancer, brain cancer, cervicocerebral cancer, esophageal cancer, thyroid cancer, stomach cancer, gallbladder cancer, liver cancer, choriocarcinoma, uterus body cancer, uterocervical cancer, kidney cancer, bladder cancer, testicular cancer, skin cancer, neuroblastoma, osteosarcoma, Ewing's sarcoma, leukemia, Hodgkin's lymphoma, acute myeloid leukemia, diffuse large B-cell lymphoma, and head and neck cancer. In some embodiments, the cancer is pancreatic cancer. In some embodiments, the cancer is ovarian cancer. In some embodiments, the cancer is platinum resistant ovarian cancer. In some embodiments, the cancer is endometrial cancer. In some embodiments, the cancer is breast cancer.

### WEE1 Inhibitors

Adavosertib has the chemical name 2-allyl-(1-[6-(1-hydroxy-1-methylethyl)pyrindin-2-yl]-6-{[4-(4-methylpiperazin-1-yl)phenyl]amino}-1,2-dihydro-3H-pyrazolo[3,4-d]pyrimidin-3-one and the following chemical structure:

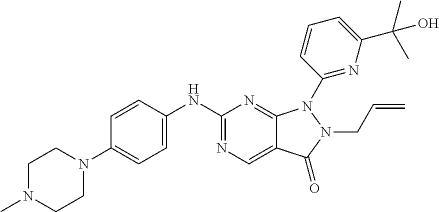 [SMILES: C=CCn1c(=O)c2cnc(Nc3ccc(N4CCN(C)CC4)cc3)nc2n1-c1cccc(C(C)(C)O)n1]

Adavosertib's activity as an inhibitor of WEE1, utility in treating various cancers, and synthesis are described in U.S. Pat. No. 7,834,019. Various crystalline forms of adavosertib are described in U.S. Pat. Nos. 8,703,779 and 8,198,281. In some embodiments, the WEE1 inhibitor administered in methods described herein is adavosertib or a pharmaceutically acceptable salt thereof. In some embodiments, the WEE1 inhibitor administered in methods described herein is adavosertib.

3-(2,6-dichlorophenyl)-4-imino-7-[(2′-methyl-2′,3′-dihydro-1′H-spiro[cyclopropane-1,4′-isoquinolin]-7′-yl)amino]-3,4-dihydropyrimido[4,5-d]pyrimidin-2(1H)-one is a WEE1 inhibitor with the following chemical structure:

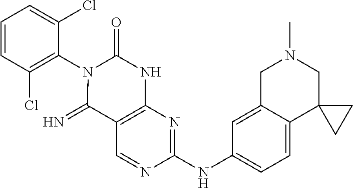 [SMILES: CN1Cc2cc(Nc3ncc4c(=N)n(-c5c(Cl)cccc5Cl)c(=O)[nH]c4n3)ccc2C2(CC2)C1]

3-(2,6-dichlorophenyl)-4-imino-7-[(2′-methyl-2′,3′-dihydro-1′H-spiro[cyclopropane-1,4′-isoquinolin]-7′-yl)amino]-3,4-dihydropyrimido[4,5-d]pyrimidin-2(1H)-one's activity as an inhibitor of WEE1, utility in treating cancer, and synthesis are described in U.S. Pat. No. 8,436,004. In some embodiments, the WEE1 inhibitor administered in methods described herein is 3-(2,6-dichlorophenyl)-4-imino-7-[(2′-methyl-2′,3′-dihydro-1′H-spiro[cyclopropane-1,4′-isoquinolin]-7′-yl)amino]-3,4-dihydropyrimido[4,5-d]pyrimidin-2(1H)-one.

### DNA-Damaging Agents

As used herein, a “DNA-damaging agent” or “DDA” is a cancer treatment that functions by causing damage to the DNA of cancer cells. DDAs act via a variety of mechanisms, including DNA crosslinking, interference with DNA replication, and inhibition of DNA synthesis. Non-limiting examples of DDAs that may be used in the methods described herein include gemcitabine, etoposide, cisplatin, carboplatin, oxaliplatin, picoplatin, methotrexate, doxorubicin, daunorubicin, 5-fluorouracil, irinotecan, mitomycin, temozolomide, topotecan, camptothecin, epirubicin, idarubicin, trabectedin, capecitabine, bendamustine, fludarabine, hydroxyurea, trastuzumab deruxtecan, and pharmaceutically acceptable salts thereof.

### Combination Therapies

In some embodiments, WEE1 inhibitors and DDAs co-administered in the methods disclosed herein are co-administered with one or more additional cancer therapies. A physician is capable of determining the one or more additional cancer therapies to co-administer to a patient depending on the particular characteristics of the patient and cancer being treated. The one or more additional cancer therapies may be administered concurrent with, prior to, or after administration of the WEE1 inhibitor and DDAs according to the methods described herein. In some embodiments, the one or more additional cancer therapies are selected from ionizing radiation, tubulin interacting agents, kinesin spindle protein inhibitors, spindle checkpoint inhibitors, poly(ADP-ribose) polymerase inhibitors, matrix metalloproteinase inhibitors, protease inhibitors, proteasome inhibitors, Bcl-2 inhibitors, heat shock protein modulators, histone deacetylase inhibitors, antiestrogens, selective estrogen receptor modulators, antiandrogens, LHRH agonists, 5α-reductase inhibitors, cytochrome P450 C17 lyase inhibitors, aromatase inhibitors, EGFR kinase inhibitors, dual erbB1 and erbB2 inhibitors, ABL kinase inhibitors, VEGFR-1 inhibitors, VEGFR-2 inhibitors, polo-like kinase inhibitors, aurora kinase inhibitors, JAK inhibitors, c-MET kinase inhibitors, cyclin-dependent kinase inhibitors, PI3K inhibitors, and mTOR inhibitors.

### EXAMPLES

The examples provided below further illustrate and exemplify the present disclosure and do not limit in any way the scope of the claims.

### Example 1: Development of an FFPE IHC Assay that is Specific for SLFN11 and Characterization of DU145 SLFN11 KO Cell Lines

### Methods

Knockout of SLFN11 in DU145 prostate cancer cells was performed by CRISPR/Cas9. sgRNAS targeting SLFN11 in exon 4 (GCGTTCCATGGACTCAAGAGAGG, protospacer adjacent motif bolded) were designed with in-house CRISPR3 software, synthesized by Integrated DNA Technology (IDT), and cloned into a vector containing CAS9 and a GFP cassette (azPGE02-Cas9-T2A-GFP). The vector was subsequently transfected into DU145 prostate cancer cells using Lipofectamine 3000 (Thermofisher Scientific). After 48 hours, cell pools with the highest green fluorescent protein (GFP) expression were single cell sorted into 96-well plates. Clones that had lost their wild-type allele were expanded to obtain cell lines from single clones. Two SLFN11-deficient clones were profiled and selected for pharmacological studies (clone KO1 and clone K02). Cell lysates from SLFN11-proficient (wt) and from SLFN11-deficient (KO1 and K02) were prepared and analyzed by standard SDS-PAGE immunoblotting. The antibodies used for immunoblotting detection were: anti-SLFN11 antibody (ab121731, 1:1000, Abcam) and, as loading control, anti-GAPDH antibody (14C10, 1:2000, CST).

DU145 (SLFN11-proficient) and HT29 (SLFN11-deficient) xenografts were grown according to the AstraZeneca Global Bioethics policy, UK Home Office legislation and the Animal Scientific Procedures Act 1986 (ASPA). SLFN11 immunohistochemistry was performed on 4 μM thick tumor sections of formalin fixed paraffin embedded tissues and carried out on Bond RX (Leica Microsystems) using ER1 antigen retrieval. Slides were stained with primary rabbit polyclonal anti-SLFN11 antibody (Abcam, ab121731) at 0.5 μg/ml for sections from xenograft tissue and at 2.5 μg/ml for sections from human tissue. Digital slides were acquired with the Aperio AT2 scanner (Leica) using a 20× objective.

### Results

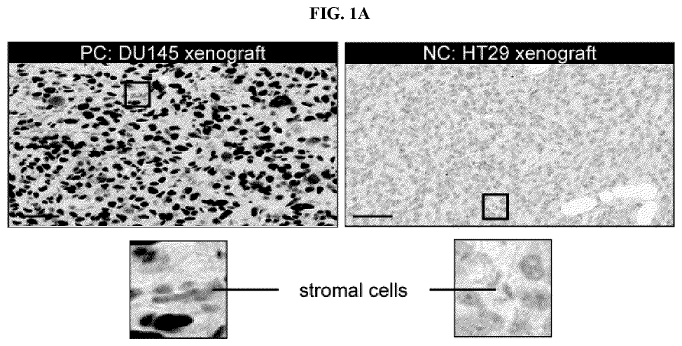

SLFN11 immunohistochemistry of SLFN11-positive DU145 and SLFN11-negative HT29 tissue confirmed the respective presence and absence of SLFN11 in these two models (FIG. 1A).

### Example 2: Resistance to DDA in DU145 SLFN11 KO Cells can be Reversed by Combination Treatment with a WEE1 Inhibitor

### Methods

Adavosertib was synthesized at AstraZeneca. Gemcitabine, cisplatin, hydroxyurea (HU), and etoposide were obtained from Tocris, and camptothecin from Sigma. Stock solutions of gemcitabine (50 mM), cisplatin (1.67 mM) and HU (1M) were prepared in aqueous solution; all other drugs were dissolved at 10 mM concentration in dimethylsulfoxide (DMSO) (10 mM).

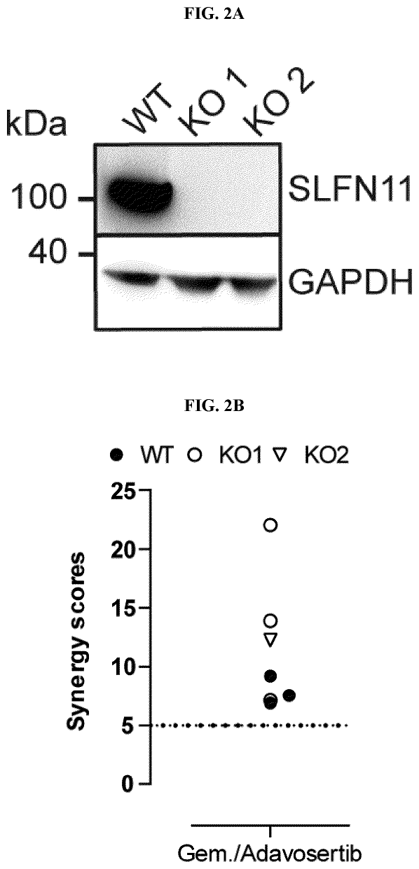

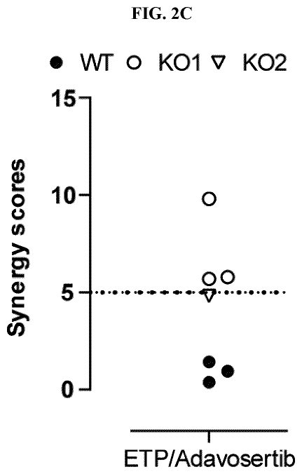

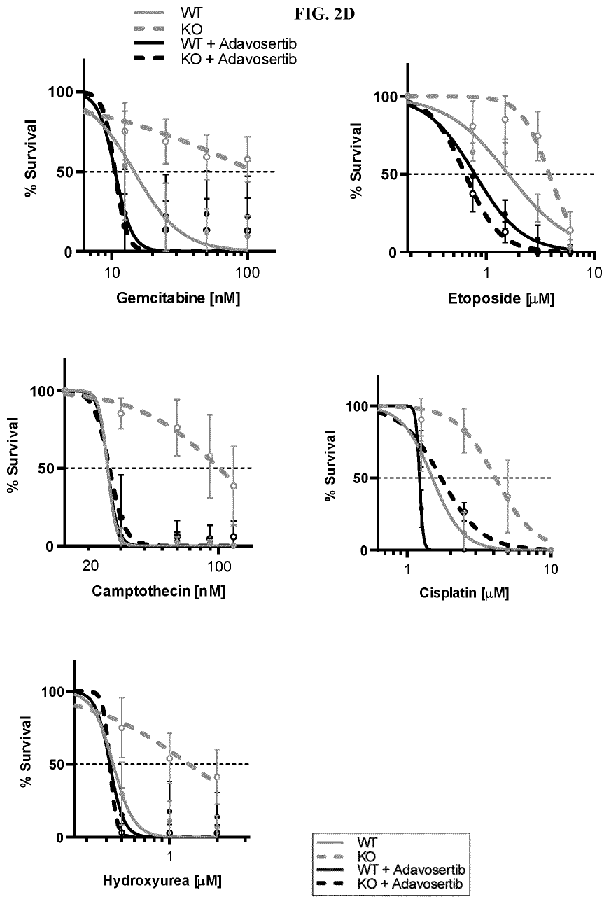

DU145 isogenic cells (WT and SLFN11 KO) were seeded in 384-well plates and allowed to settle overnight. FIG. 2A shows immunoblots for SLFN11 WT and KO DU145 isogenic cells used in the experiments. KO 1 and KO 2 were two different CRISPR-KO clones. Cells were dosed with compound solutions in a 6×6 concentration matrix, with top doses of 3 adavosertib, 0.1 μM gemcitabine, and 1 μM etoposide, using an Echo 555 (LabCyte). Five days following continuous treatment, cell viability was determined by live-dead SyTox green assay (Life Technologies, Carlsbad, Calif., USA). The number of live cells was calculated by subtracting the dead and total reads. Using this methodology, cell numbers per well were also determined at the point of treatment (day 0). Data are shown using the equation [1−(Ti−Tz)/(C−Tz)]×100 for values for which Ti≥Tz and [1−(Ti−Tz)/Tz]×100 for concentrations for which Ti<Tz,×100, where Ti=compound-treated cells; Tz=cells at 0 h time point and C=control cells. This gives a 0-200% scale of live cell number, where 0-100% represents growth inhibition and 100-200% represents cell killing.

Combination activity (synergism) was calculated using the Loewe dose-additivity model in Genedata Screener (Genedata, Basel, Switzerland) software. This model calculates the expected result if the effects of the two compounds were additive based upon the two monotherapies. The excess score reflects how much above the predicted additive effect the experimental result is. The program provides a synergy score for the combination, which reflects both the strength of the excess score, and the dose dependency. A score >5 is deemed synergistic.

For cell survival experiment in 96 well plate, cells were seeded in 96-well plates, following compound dosing using a HP dispenser. 72 hours later, cell viability was determined with end-point CellTiter-Glo luminescent assays (Promega). Percentage growth was calculated using the equation (T−T0)/(C−T0)×100, where T=compound-treated cells; T0=cells at 0 h time point and C=control cells. Dose response curves were plotted in GraphPad prism.

### Results

Combination treatment with adavosertib and gemcitabine or etoposide consistently produced higher synergy scores in SLFN11 KO cells when compared to wild-type, SLFN11-proficient cells (FIGS. 2B and 2C, respectively). The higher synergy scores indicate that the combination treatments with a WEE1 inhibitor and a DDA are more effective in SLFN11 KO cells relative to wild-type cells, relative to the effect of the monotherapies with either agent. The combination synergy experiment was validated by lower throughput assay formats. The results are shown in FIG. 2D for combination of different indicated DDAs (gemcitabine, etoposide, camptothecin, cisplatin, and hydroxyurea) with adavosertib. In all cases, SLFN11 KO cells (dotted grey lines) were found resistant to each of the DDAs when compared to wild type cells (continuous grey lines). Combination of DDA with adavosertib did not add significant antiproliferative effect in the SLFN11-proficient cells (solid black lines). In SLFN11 KO cells, however, the same combinations led to a significant curve-shift compared to the DDA monotherapy in SLFN11 deficient cells (shown in dotted black line), confirming that these cells can be completely re-sensitized to DDA treatment by co-administering adavosertib.

### Example 3: Resistance to Gemcitabine in SLFN11-Deficient Cell Lines can be Reversed by Combination Treatment with a WEE1 Inhibitor

### Methods

SLFN11 RNA seq data (log 2 RPKM values) were downloaded from cancer cell line encyclopedia (CCLE) (Barretina J. et al., Nature, 2012; 483: 603-607) and drug response data (log(IC₅₀) and area under the dose-response curve (AUCs)) from drug sensitivity in cancer database (Yang W et al., Nucleic Acids Res, 2013; 41: D955-61). Cell lines with CCLE RNA seq log 2 RPKM values below 1 were defined as SLFN11-deficient and cell lines with log 2 RPKM values above 1 as SLFN11-proficient. Nineteen pancreatic cell lines in 384-well plates were dosed with increasing concentrations of adavosertib and gemcitabine in a 6×6 concentration matrix using an Echo 555 (LabCyte). The dose range was 0-3 μM for adavosertib, and 0-0.3 μM for gemcitabine; in both cases dilutions 1:3 from the top dose were performed. Five days following continuous treatment, cell viability was determined by live-dead SyTox green assay (Life Technologies, Carlsbad, Calif., USA). Synergy was analyzed in Genedata screener software using the Loewe dose-additivity model as described above.

### Results

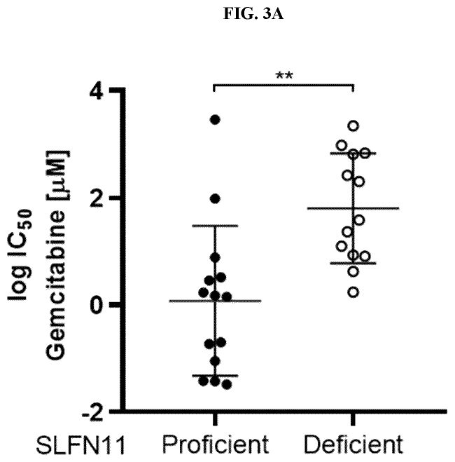

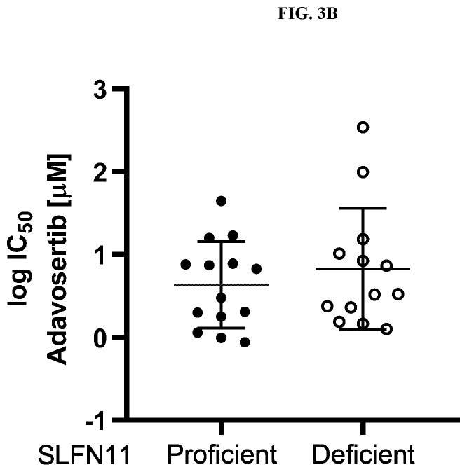

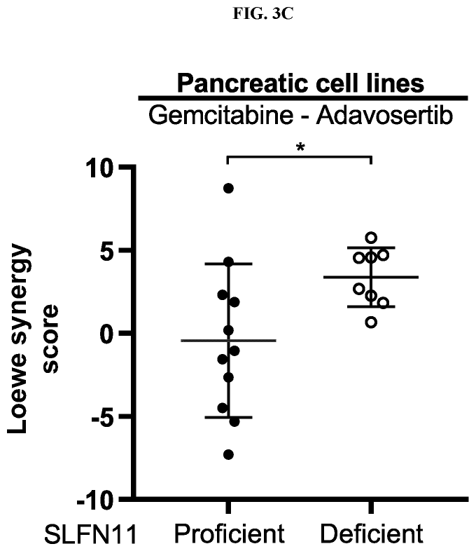

The results presented in Example 2 were validated in a panel of pancreatic cancer cell lines. In this panel, upon dose response treatments with gemcitabine monotherapy, SLFN11-deficient cell lines were found on average 100 times less sensitive than the SLFN11-proficient cells (FIG. 3A). SLFN11-deficient and SLFN11-proficient pancreatic cancer cell lines showed the same response to adavosertib monotherapy treatment (FIG. 3B). However, combination treatment with gemcitabine and adavosertib was significantly more synergistic in SLFN11-deficient than SLFN11-proficient pancreatic cancer cells (FIG. 3C). The results indicate that combination therapy with a WEE1 inhibitor and a DDA is expected to be more effective in patients with SLFN11-deficient cancer cells compared to monotherapy with the WEE1 inhibitor or DDA.

1. A method of treating cancer in a patient comprising:
a) selecting a patient diagnosed with cancer;
b) determining whether the patient's cancer cells are SLFN11-deficient; and,
c) if the patient's cancer cells are SLFN11-deficient, co-administering a WEE1 inhibitor and a DNA-damaging agent to the patient.

2. A method of treating cancer in a patient comprising:
a) selecting a patient diagnosed with cancer;
b) determining whether SLFN11 expression is lower in the patient's cancer cells relative to the patient's SLFN11-expressing non-cancer cells; and,
c) if SLFN11 expression is lower in the patient's cancer cells relative to the patient's SLFN11-expressing non-cancer cells, co-administering a WEE1 inhibitor and a DNA-damaging agent to the patient.

3. The method of claim 1 or 2, wherein the patient's cancer cells are negative for SLFN11 expression.

4. The method of any one of claims 1 to 3, wherein the expression level of SLFN11 is determined by immunohistochemistry, mass spectrometry, in-situ hybridization, NanoString, reverse transcription quantitative polymerase chain reaction (RT-qPCR), microarray analysis, bisulfite sequencing, or quantitative methylation-specific polymerase chain reaction (Q-MSP).

5. The method of any one of claims 1 to 3, wherein the expression level of SLFN11 is determined by immunohistochemistry.

6. A method of treating cancer in a patient comprising:
a) selecting a patient diagnosed with cancer;
b) determining the expression level of SLFN11 in the patient's cancer cells; and,
c) if the expression level of SLFN11 is <10%, co-administering a WEE1 inhibitor and a DNA-damaging agent to the patient.

7. The method of claim 6, wherein the expression level of SLFN11 is 0%.

8. The method of claim 6 or 7, wherein the expression level of SLFN11 is determined by immunohistochemistry, mass spectrometry, in-situ hybridization, NanoString, reverse transcription quantitative polymerase chain reaction (RT-qPCR), microarray analysis, bisulfite sequencing, or quantitative methylation-specific polymerase chain reaction (Q-MSP).

9. The method of claim 6 or 7, wherein the expression level of SLFN11 is determined by immunohistochemistry.

10. A method of treating cancer in a patient that is resistant to treatment with a DNA-damaging agent, comprising:
a) determining whether the patient's cancer cells are SLFN11-deficient; and,
b) if the patient's cancer cells are SLFN11-deficient, co-administering a WEE1 inhibitor with the DNA-damaging agent to the patient.

11. A method of treating cancer in a patient that is resistant to treatment with a DNA-damaging agent, comprising:
a) determining whether SLFN11 expression is lower in the patient's cancer cells relative to the patient's SLFN11-expressing non-cancer cells; and,
b) if SLFN11 expression is lower in the patient's cancer cells relative to the patient's SLFN11-expressing non-cancer cells, co-administering a WEE1 inhibitor with the DNA-damaging agent to the patient.

12. The method of claim 10 or 11, wherein the patient's cancer cells are negative for SLFN11 expression.

13. The method of any one of claims 10 to 12, wherein the expression level of SLFN11 is determined by immunohistochemistry, mass spectrometry, in-situ hybridization, NanoString, reverse transcription quantitative polymerase chain reaction (RT-qPCR), microarray analysis, bisulfite sequencing, or quantitative methylation-specific polymerase chain reaction (Q-MSP).

14. The method of any one of claims 10 to 12, wherein the expression level of SLFN11 is determined by immunohistochemistry.

15. A method of treating cancer in a patient that is resistant to treatment with a DNA-damaging agent, comprising:
a) determining the expression level of SLFN11 in the patient's cancer cells; and,
b) if the expression level of SLFN11 is <10%, co-administering a WEE1 inhibitor with the DNA-damaging agent to the patient.

16. The method of claim 15, wherein the expression level of SLFN11 is 0%.

17. The method of claim 15 or 16, wherein the expression level of SLFN11 is determined by immunohistochemistry, mass spectrometry, in-situ hybridization, NanoString, reverse transcription quantitative polymerase chain reaction (RT-qPCR), microarray analysis, bisulfate sequencing, or quantitative methylation-specific polymerase chain reaction (Q-MSP).

18. The method of claim 15 or 16, wherein the expression level of SLFN11 is determined by immunohistochemistry.

19. The method of any one of claims 1 to 18, wherein the cancer is selected from the group consisting of pancreatic cancer, endometrial cancer, ovarian cancer, melanoma, lung cancer, colorectal cancer, colon cancer, rectal cancer, prostate cancer, breast cancer, brain cancer, cervicocerebral cancer, esophageal cancer, thyroid cancer, stomach cancer, gallbladder cancer, liver cancer, choriocarcinoma, uterus body cancer, uterocervical cancer, kidney cancer, bladder cancer, testicular cancer, skin cancer, neuroblastoma, osteosarcoma, Ewing's sarcoma, leukemia, Hodgkin's lymphoma, acute myeloid leukemia, diffuse large B-cell lymphoma, and head and neck cancer.

20. The method of any one of claims 1 to 18, wherein the cancer is ovarian cancer.

21. The method of any one of claims 1 to 18, wherein the cancer is platinum resistant ovarian cancer.

22. The method of any one of claims 1 to 18, wherein the cancer is endometrial cancer.

23. The method of any one of claims 1 to 18, wherein the cancer is pancreatic cancer.

24. The method of any one of claims 1 to 18, wherein the cancer is breast cancer.

25. The method of any one of the preceding claims, wherein the DNA-damaging agent is selected from the group consisting of gemcitabine, etoposide, cisplatin, carboplatin, oxaliplatin, picoplatin, methotrexate, doxorubicin, daunorubicin, 5-fluorouracil, irinotecan, mitomycin, temozolomide, topotecan, camptothecin, epirubicin, idarubicin, trabectedin, capecitabine, bendamustine, fludarabine, hydroxyurea, trastuzumab deruxtecan, and pharmaceutically acceptable salts thereof.

26. The method of any one of the preceding claims, wherein the DNA-damaging agent is selected from the group consisting of gemcitabine, etoposide, camptothecin, cisplatin, hydroxyurea, and pharmaceutically acceptable salts thereof.

27. The method of any one of the preceding claims, wherein the DNA-damaging agent is gemcitabine or a pharmaceutically acceptable salt thereof.

28. The method of any one of claims 1 to 25, wherein the DNA-damaging agent is trastuzumab deruxtecan.

29. The method of any one of the preceding claims, wherein the WEE1 inhibitor is adavosertib or a pharmaceutically acceptable salt thereof.

30. The method of any one claims 1 to 24, wherein the DNA-damaging agent is gemcitabine or a pharmaceutically acceptable salt thereof, and the WEE1 inhibitor is adavosertib or a pharmaceutically acceptable salt thereof.

31. The method of any one of claims 1 to 24, wherein the DNA-damaging agent is trastuzumab deruxtecan, and the WEE1 inhibitor is adavosertib or a pharmaceutically acceptable salt thereof.

32. The method of claim 30, wherein 175 mg adavosertib is administered to the patient on days 1, 2, 8, 9, 15, and 16, and 800 mg/m² gemcitabine or a pharmaceutically acceptable salt thereof is administered to the patient on days 1, 8, and 15 on a 28-day cycle.

33. The method of claim 30, wherein 175 mg adavosertib is administered to the patient on days 1, 2, 8, 9, 15, and 16, and 1,000 mg/m² gemcitabine or a pharmaceutically acceptable salt thereof is administered to the patient on days 1, 8, and 15 on a 28-day cycle.
